(19) United States
(12) Patent Application Publication
(10) Pub. No.: US 2009/0035835 A1
(43) Pub. Date: Feb. 5, 2009

(54) Method and device for producing biomass of photosynthesizing microorganisms/phototrophical algae and biomass of these microorganisms pigments

(76) Inventor: Vladimir Slavin, Ashqelon (IL)

Correspondence Address: Vladimir Slavin, 32, Ben-Gurion str., apt. 24, Ashqelon, 78290, IL

(21) Appl. No.: 12/221,564

(22) Filed: Aug. 4, 2008

(30) Foreign Application Priority Data
Jul. 31, 2007 (IL) 184941

(51) Int. Cl. C12N 13/00; C12M 1/42; C12R 1/89

(52) U.S. Cl. 435/173.8; 435/292.1

(57) ABSTRACT

The invention relates to biotechnology, in particular to methods and means of physical action on biological structures of photosynthesing microorganisms, phototrophic algae in particular. The invention can be used in pharmaceutical, cosmetic and foodstuff industries, as well as for obtaining biofuel from algae In the process of the method implementation radiation of cultivated solution of photosynthesizing microorganisms/phototrophical algae is carried out by the action of electromagnetic waves of mm range and low intensity. Stimulation of increasing photosynthesizing microorganisms/phototrophical algae biomass and biomass of their pigments (excretions) is obtained with industrial production as a result of the resonance effect which is caused by the interaction of electromagnetic wave and biological cell. Irradiation of cultivated solution of photosynthesizing microorganisms/phototrophical algae is performed by electromagnetic waves of mm range and low intensity at different phases of cultured biological objects development.

### FIELD OF THE INVENTION

The invention relates to biotechnology, in particular to methods and means of physical action on biological structures of photosynthesing microorganisms, phototrophic algae in particular. The invention can be used in pharmaceutical, cosmetic and foodstuff industries, as well as for obtaining biofuel from algae.

### BACKGROUND OF THE INVENTION

Mariculture is one of the fast growing directions of marine biotechnology. This direction deals with commercial cultivation of valuable invertebrates and algae. Algae cultivation has resulted in great achievements, although it has a history of only a few decades. With higher productivity and resulting higher profit, it has become the leading marine exploitation industry with the brightest prospects. The process of cultivation in mariculture consists of two steps. The first step is deriving of invertebrates' juveniles and algae seedlings find second step is their cultivation up to a crop deriving. [1-8].

There exist inventions which give methods of producing pigments which are present in membranes of photosynthesizing microorganisms. Now methods of increasing photosynthesizing microorganisms (phototrophic algae) biomass (besides application of solar energy) using UV and laser radiation (blue, green or red range of visible light) on microorganisms are known [7-30].

Patent WO 1991/005849 “The method of cultivation of photosynthetic microorganisms and cells of photosynthetic macrophytes” [24] can be considered as an analogue to the proposed invention.

“This method includes the following stages: a) pretreating water source for functioning as a culture medium; b) circulating said pretreated cultured water through a closed, continuous conduit which is transparent to sunlight, disposed on a floor substantially horizontally within a protective growth module, which is also transparent to sunlight; c) inoculating the said circulating cultured water with cells of photosynthetic species; d) monitoring one or more physical, chemical or biological characteristics of the said circulating cultured water containing photosynthetic cells within the said closed conduit; e) automatically comparing the value of the said observed physical, chemical or biological characteristic of the said circulating cultured water to a predetermined optimum value for said characteristic; f) automatically adjusting the said physical, chemical or biological characteristic of the said circulating cultured water when the said observed value deviates from the said optimum value; g) isolating a concentrated portion of photosynthetic cells grown in the said conduit from the said circulating cultured water without affecting its total volume; h) separating and recovering said photosynthetic cells from the said circulating cultured water. Disadvantages of this method are connected with large volumes of water and raw materials consumed, considerable quantity of energy used, dependence of the quantity obtained on spectral components of light flow and the period of strain culture light exposure, high cost of the end product. As an expert has shown, end product (algae pigment) has the greatest value for producing (manufacture) of medical, cosmetic, food and feed preparations.”

The solution closest to that proposed in our patent is given in U.S. Pat. No. 7,080,478 “Technology for cultivation of Porphyra and other algae in land-based sea water ponds” (2006) [29]. This invention provides technology and systems of cultivating different types of algae in land-based seawater ponds having a climatically suitable and nutrient controlled environment. These land-based ponds may be built in any part of the world with structural engineering and architectural modifications. The invention provides methods of designing different stages of growth, and defining the special conditions to optimize each of the different stages in controlled environments. The technology includes techniques of enriching the algae with desired nutrients and ingredients for the production of high quality products that are free. According to the goals mentioned above, the aims of the proposed invention consist in presenting a method and device for intensive industrial cultivation of photosynthesizing microorganisms and production of their pigments with simultaneous decrease in consumption of raw material volumes, water and electric power.

As experiments have shown [31-40], intensity in photosynthesizing microorganisms (phototrophic algae) and pigment biomass increase depend on the values of spectral components of light and additional irradiation of photosynthesizing microorganisms (phototrophic algae) by electromagnetic waves of millimeter range.

It is known that photosynthesizing microorganisms (phototrophic algae) use solar light energy for its growth and for partial provision of energy for intracellular processes of biosynthesis. Electromagnetic irradiation of photosynthesizing microorganisms (phototrophic algae) by electromagnetic waves of mm range stimulates cellular metabolism, also increasing potential of mitochondrial membranes, ATP synthesis and oxygen consumption. In plasmatic membranes, depending on the irradiation wavelength, photo-induced change of proton gradient and activation of ion channels are being observed, thus leading to pH change, redistribution of calcium ions and stimulation of metabolism and cell division. As a result, there occurs increase in total quantity of cell biomass obtained during cultivation and the quantity of pigment in cells.

Industrial pigment production is absent at the present time.

According to the goals mentioned above, the aims of the proposed invention consist in presenting a method and device for intensive industrial cultivation of photosynthesizing microorganisms (phototrophic algae) and production of their pigments with simultaneous decrease in consumption of raw material volumes, water and electric power.

### SUMMARY OF THE INVENTION

The aims set forth are attained by the technical solution proposed according to which sea water or water from a saline reservoir containing photosynthesizing microorganisms (phototrophic algae) after corresponding water treatment enters the bioreactor where photosynthesizing microorganisms (algae, bacteria) cultivation occurs, and additional irradiation by electromagnetic waves of mm range and low intensity is carried out together with natural sun lighting; then sorting of photosynthesizing microorganisms (phototrophic algae) biomass is performed according to aging criterion. Mature photosynthesizing microorganisms (phototrophic algae) obtained after sorting enter a microorganism stress bioreactor (stress tank) where mature photosynthesizing microorganisms (algae, bacteria) biomass under simultaneous non-radioactive irradiation by electromagnetic waves of mm range and low intensity is submerged into bidistilled water (or other substance) preliminary irradiated by electromagnetic waves of mm range and low intensity. After that the solution of photosynthesizing microorganisms (phototrophic algae) purple membranes goes from stress bioreactor to centrifugation/flotation of photosynthesizing microorganisms (algae, bacteria) pigment resulting in isolation of pigment molecules which then proceed to drying, during and after which pigment molecules are irradiated by electromagnetic waves of mm range and low intensity. Further, pigment molecules go to the block of output control and then to the block of packing into waterproof material for delivering to customers. A variant of water treatment is possible where water from a water treatment block output is delivered to a separator of photosynthesizing microorganisms (algae, bacteria) selection, after that the solution goes to cultivation and the discharge from the separator of photosynthesizing microorganisms (phototrophic algae) selection is directed to the output of discharge from the post of water intake. Besides, the discharge after separator of photosynthesizing microorganisms (phototrophic algae) selection is directed to bioreactor of bacteria cultivation, and discharge after centrifugation goes to the block of output control.

The device for implementing the method consists of a post of water intake, water treatment block, bioreactors of microorganisms stress and cultivation, generators of electromagnetic radiation, block of raw materials supply, bidistilled water (or other substance) source, centrifugation block, drying block, packaging block, control block.

Besides, the device for implementing the method includes additionally a separator for photosynthesizing microorganisms (phototrophic algae) selection, a separator for photosynthesizing microorganisms (phototrophic algae) sorting, generators for non-radioactive radiation of electromagnetic waves and low intensity. In this case the output of the post of water intake is connected to the input of water treatment block and main output of the latter is connected to the input of separator for photosynthesizing microorganisms (algae, bacteria) selection and the output of this separator is connected to the input of bioreactor of photosynthesizing microorganisms (phototrophic algae) cultivation which, in its turn, is connected to the output of the raw material supply block and generator for non-radioactive radiation of electromagnetic waves with mm range and low intensity.

Output of bioreactor for photosynthesizing microorganisms (phototrophic algae) cultivation is connected to the input of separator for photosynthesizing microorganisms (algae, bacteria) sorting, its main output being connected with the input of microorganisms stress bioreactor which is connected to a source of bidistilled water (or other substance) and generator for non-radioactive radiation of electromagnetic waves with mm range and low intensity. Besides, output of microorganism stress bioreactor is connected to the block of centrifuges for isolating photosynthesizing microorganisms (algae, bacteria) pigment and its main output is connected to the drying block which is connected to generator for non-radioactive radiation of electromagnetic waves with mm range and low intensity and has its main output connected to the input of the block of output control. Block of output control is also connected to generator for non-radioactive radiation of electromagnetic waves with mm range and low intensity, and output of the block of output control is connected to the input of packaging block.

Control block of the device carries out optimization of photosynthesizing microorganisms (algae, bacteria) cultivation process and production of photosynthesizing microorganisms (algae, bacteria) pigments. Inputs of the control block are connected to outputs of control and measuring devices at the post of water intake, water treatment block, raw material supply block, bioreactors of microorganism stress and bacteria cultivation, generators for non-radioactive radiation of electromagnetic waves and low intensity, separator of photosynthesizing microorganisms (algae, bacteria) selection, separator of photosynthesizing microorganisms (algae, bacteria) sorting, block of centrifuges for isolating photosynthesizing microorganisms (algae, bacteria) pigment, drying block, block of output control and packaging block. Current values of controlled parameters of the device elements are introduced into models of processes for bacteria cultivation and microorganism stress and their outputs are connected to the block of decision taking, the outputs of which are connected to control outputs in control block.

In this case control outputs of the control block are connected to inputs of control schemes at the post of water intake, water treatment block, raw material supply block, bioreactors of microorganism stress and bacteria cultivation, generators for non-radioactive radiation of electromagnetic waves and low intensity, separator of photosynthesizing microorganisms (algae, bacteria) selection separator of photosynthesizing microorganisms (algae, bacteria) sorting, block of centrifuges for isolating photosynthesizing microorganisms (algae, bacteria) pigment, drying block, block of output control and packaging block. As experiments in cultivating photosynthesizing microorganisms biomass (algae, bacteria) have shown the increase (in 2-3 times) as well as biomass of their pigments (in 3-5 times), it is necessary to carry out the following additional operations:

photosynthesizing microorganisms irradiate on frequencies in a band of absorption of electromagnetic waves with atomic oxygen, hydrogen and/or water vapor on the certain phases of microorganisms cultivation,

parameters of additional electromagnetic radiation change during time for each phase of cultivation.

### BRIEF DESCRIPTION OF THE DRAWINGS

The invention is illustrated by the drawings attached wherein:

FIG. 1 shows the block-scheme of the device for producing photosynthesizing microorganisms biomass, mainly halobacteria Halobacterium, and pigment biomass of the said microorganisms, bacteriorhodopsin in particular, according to the solution proposed.

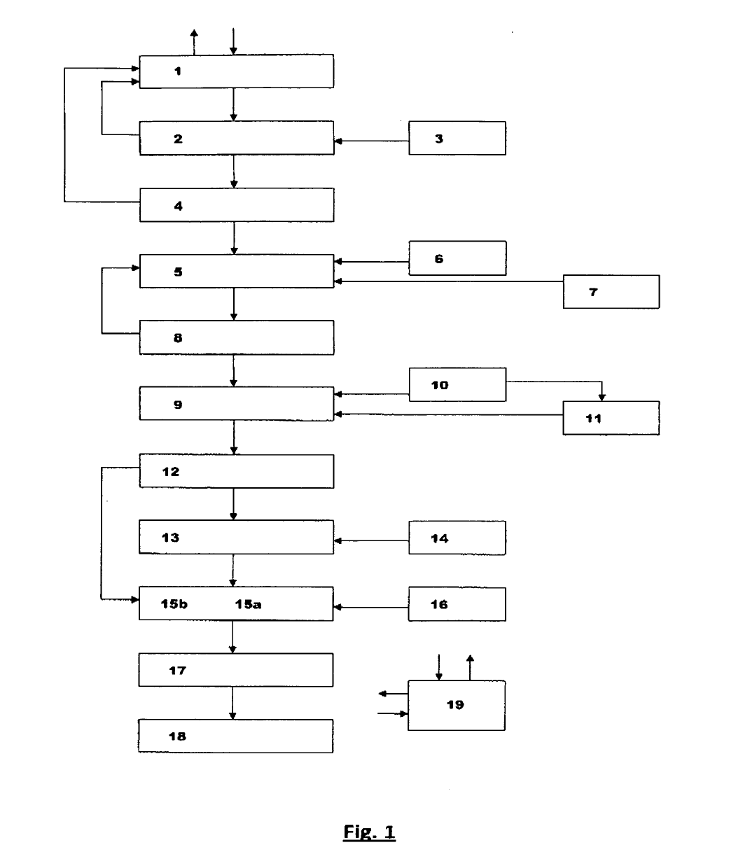

FIG. 2 shows conventional connections of the output control block of the device.

### DETAILED DESCRIPTION OF THE PREFERRED EMBODIMENTS

The method proposed and the device for its implementation have been briefly described in the section “Summary of the invention”.

The present section presents full description of the solution proposed disclosing preferred embodiments and referring to figure numbers which clarify the invention.

With cosmic electromagnetic waves propagation reaching the Earth's biosphere, the energy of these waves is being absorbed. Electromagnetic waves absorption can be divided into resonance and non-resonance absorption. Non-resonance absorption of electromagnetic waves means transforming the energy of radio-waves into thermal energy of the environment. With resonance absorption of electromagnetic waves, radio-wave energy is used for moving substance molecules up to higher energy levels.

Non-resonance absorption of electromagnetic waves can occur with radio-waves propagation in feeder lines of receiving and transmitting equipment due to resulting conductivity of coaxial cables, waveguides, etc.; due to radio-wave energy consumption for overcoming mutual friction of gas molecules possessing electric and magnetic moments, as well as due to particles of hydrometeors (rain, hail, etc.) with electromagnetic waves propagation in troposphere.

Resonance absorption of electromagnetic waves in troposphere is stipulated by transition of the medium (gas) molecules up into higher energy levels at the expense of radio-wave energy. It attains its maximum when wave frequency coincides with one of the frequencies of allowed quantum transitions. Atmosphere absorption leads to cosmic radiation attenuation, to decreasing the energy flow density of cosmic electromagnetic waves. Absorption of electromagnetic waves is manifested at frequencies higher than 10 Hz in troposphere.

In this case main absorption of cm and mm waves is caused by oxygen (resonance absorption bands in the vicinity of frequencies 60 and 129 GHz) and water vapor (absorption bands in the vicinity of 22 and 183 GHz). At present it is generally recognized that each of the electromagnetic waves ranges reaching the Earth's biosphere has played a definite role in the evolution of the living nature. There exist theories according to which electromagnetic fields in biological systems play regulatory and informative roles.

Hereby it is considered that electromagnetic fields quanta act as material carriers of information flows in cellular type biospheres. Electromagnetic field flows inside biosystems form information basis of their vital activity (rhythm), and outer electromagnetic field flows serve as factors which to a certain extent regulate inner information flows.

It is believed that biological systems appeared to be poorly adapted to the action of electromagnetic waves within the given ranges of cosmic radiation during million years of evolution on the Earth. Experiments in Russia, the Ukraine and Israel have shown that electromagnetic waves of the mm range can cause response of biological systems even with low non-thermal intensity.

Thus, biological effect of mm waves influence on biological systems has been found out as well as its threshold character in relation to strength. Minimum action strength equal to 5-10 mcWatt/cm² has been found out and the value of threshold strength had been theoretically calculated as close to the natural background in the given range and being equal to 10⁻¹⁹ W/m²×Hz [41-58]. This permits to apply artificial mm radiation as a factor of physiological regulation of cell metabolism in photosynthesizing microorganisms (phototrophic algae) under industrial conditions of their cultivation.

Numerous experiments [41-58] have shown, that the small quantity of unitary absorbed energy causes both at proeucaryotical and at eucaryotical photosynthetic organisms such rather essential consequences as acceleration of growth, increase in an output of a biomass in 2-3 times, the increase in quantity of pigments in a cell up to 3-5 times, and a level of organic connections excretion on medium, that in our opinion testifies to presence self-accelerating mechanisms in development of consequences of an irradiation.

During irradiation of biological objects containing water which is present partially in the free state and partially in composition with organelles in corresponding biological systems, it is generally agreed that 50% of the absorbed dose in the “middle cell” goes for water, while the other 50% are accounted for its organelles and dissolved substances. In accordance with localization of consumed energy (in water or main substance) it is possible to discuss direct and indirect action of electromagnetic radiation.

Thus, the effect of hydrogen peroxide formed which is a strong acceptor of electrons able to regulate functioning of many fermentative systems can be considered as one of the causes of non-thermal influence of radiation in mm range on biological objects. Presence of peroxides formed could have intensified photosynthesis processes together with pigments storage what we have observed as a result of radiation in millimeter range.

The above said coincides with the point of view that photosynthetic oxygen is formed not out of water, but of hydrogen peroxide having exogenous or endogenous origin, hence increase of peroxides in the cell under the action of some factors could have corresponded to photosynthesis intensification.

Some authors believe that biological membrane is the most important point of this radiation application because low intensity of microwave radiation leads to acceleration or suppression in active ions transport, to changes in biological membranes permeability due to proteins irradiation in the cell membrane and protein component in the complex of ATP synthesis.

The majority of researchers have shown that cell membranes of different objects (from plant cells to erythrocytes) serve as the main location of influence for radiation in mm range: primary mechanisms which determine final effect of radiation in mm range influence are developed in the membranes.

Thus, electromagnetic radiation in mm range and of low intensity is capable of causing the biological systems reaction which is impossible in case of electromagnetic radiation of high intensity.

Experiments conducted in Israel, Russia and the Ukraine have shown that formation of additional radical and peroxide groups leads to stimulation of cell metabolism including increase of mitochondrial membranes potential, acceleration in ATP synthesis, thus leading to the increase of microorganisms biomass, halobacteria Halobacterium salinarium among them. Quantity of bacteriorhodopsin obtained depends on the conditions of halobacteria cultivation. Under conditions of oxygen deficiency in halobacteria purple membranes synthesis of protein bacteriorhodopsin occurs which permits halobacteria breathing due to photophosphorylation. Experiments by different scientists have proved the intensity of bacteriorhodopsin formation to depend on the value of media illumination, period of non-radioactive electromagnetic radiation of low intensity and oxygen quantity in the medium [53, 54].

Researches [67, 68, 69, 70] have shown the effect of additional THz electromagnetic radiation (100-1000 GHz) action on organic substances at frequencies of nitrogen oxide NO molecular spectrum. It can be explained by the fact that cosmic radiation when going through the Earth's atmosphere is largely absorbed by substances comprising the atmosphere (e.g. oxygen, hydrogen, nitrogen oxide, etc.).

Effectiveness of THz electromagnetic radiation action depends on duration and frequency of THz electromagnetic radiation as well as on the composition of the culture solution. Molecular spectrum of rotating-oscillatory energy transition of nitrogen oxide molecules appear in the frequency range of 150.176-150.644 GHz. Existing radiation generators overlap the range up to 100 GHz and emit signal of one type of oscillations H₁₀, E₁₁ with one polarization. However, in accordance with Dirac theory molecular spectrum represents electromagnetic oscillations of different types, polarizations and propagation directions. Due to this nitrogen oxide molecules interaction is to be more effective with electromagnetic field having the structure of radiation spectrum and absorption of this molecule, thus determining metabolic changes in pigment cells.

That's why THz electromagnetic radiation can be used for increasing pigment biomass of cultured microorganisms (phototrophic algae) at the stage of cultured microorganisms stress and further at phases of these microorganisms pigment treatment.

Different technical means can be used for imitating radiation spectra of cultured solutions. Technical means for generating electromagnetic waves absorption spectra in the atmosphere in the frequency range of 20-200 GHz as well as molecular spectra of radiation and absorption of substances comprising the pigment of cultivated solution in the frequency range of 100-1000 GHz are selected according to cosmic rays spectra obtained on Earth and reported by NASA. To attain that mode (modes) of electromagnetic radiation parameters modulation [54, 55] are determined in the frequency range mentioned; this modulation mode being chosen out of modulation modes (amplitude modulation, frequency modulation, noise modulation. modulation of radiation pulses width and frequency) or out of combinations thereof. It is necessary for better imitating the different kinds of electromagnetic signal which irradiates the surface of cultivated solution from the atmosphere. When choosing the modulation mode (modes) evaluation of imitation signal effectiveness is carried out together with economical estimation of technical realizability of signals imitator.

In accordance with the solution proposed in the present application, sea water or water from a saline reservoir containing photosynthesizing microorganisms (algae, bacteria) enters post of water intake 1, wherefrom it is pumped to water treatment block 2 where preliminary mechanical water purification (from foreign objects and suspended particles) is carried out, after that additional water irradiation by non-radioactive electromagnetic waves of mm range and low intensity is performed from radiation generator 3 resulting in formation of additional radical and peroxide groups in water. Water solution from water treatment block 2 is delivered to separator for photosynthesizing microorganisms (algae, bacteria) selection 4 out of which young photosynthesizing microorganisms (algae, bacteria) are directed to bioreactor for bacteria cultivation 5 wherein nutrients from block of raw material supply 6 are introduced and additional irradiation by non-radioactive electromagnetic waves of mm range and low intensity is performed from radiation generator 7. Discharge from water treatment block 2 is discharged into the discharge of water intake post 1. Old photosynthesizing microorganisms (algae, bacteria) (discharge from separator for photosynthesizing microorganisms (algae, bacteria) selection 4 is also discharged into discharge of water intake post 1. Photosynthesizing microorganisms (algae, bacteria) cultivation is carried out in bioreactor for bacteria cultivation 5 where together with natural sun lighting an additional irradiation of cultivated solution by non-radioactive electromagnetic waves of mm range and low intensity is performed. Cultivated solution from bioreactor for bacteria cultivation 5 enters separator for photosynthesizing microorganisms (algae, bacteria) sorting 8 where the bacteria are separated according to aging criterion. Mature photosynthesizing microorganisms (algae, bacteria) are sent from separator for photosynthesizing microorganisms (algae, bacteria) sorting 8 to microorganism stress bioreactor 9 where the biomass of mature photosynthesizing microorganisms (algae, bacteria) is submerged into bidistilled water from source 11 and is irradiated by non-radioactive electromagnetic waves of mm range and low intensity from generator 10.

Experiments conducted in Israel, Russia and Ukraine have shown that the water of cultivated solution which has been preliminary irradiated by non-radioactive electromagnetic waves of mm range and low intensity leads to increase in excretion of photosynthesizing microorganisms (algae, bacteria) pigment for 3-5 times. That's why bidistilled water enters bioreactor 9 from source 11 where it is preliminary irradiated by non-radioactive electromagnetic radiation of mm range and low intensity from generator 10. Increased excretion of photosynthesizing microorganisms (algae, bacteria) pigment occurs in bioreactor 9.

The obtained solution of photosynthesizing microorganisms (algae, bacteria) purple membranes goes from microorganism stress bioreactor 9 to block of centrifuges 12 for isolating photosynthesizing microorganisms (algae, bacteria) pigment as a result of which molecules of pigment (bacteriorhodopsin) are isolated and sent to drying block 13 where during drying and after it pigment (bacteriorhodopsin) molecules are irradiated by non-radioactive electromagnetic waves of mm range and low intensity from generator 14. Pigment (bacteriorhodopsin) molecules which have been obtained from block of centrifuges for isolating photosynthesizing microorganisms (algae, bacteria) pigment 12 are delivered from drying block 13 to block of output control 15a where pigment (bacteriorhodopsin) molecules are irradiated before packaging by non-radioactive electromagnetic waves of mm range and low intensity from generator 16. Then pigment (bacteriorhodopsin) molecules enter packaging block 17 for delivery to customers 18.

Discharge from block of centrifuges for isolating photosynthesizing microorganisms (algae, bacteria) pigment 12 is delivered to block of output control 15b. This biomaterial (discharge from block of centrifugation) is irradiated in block of output control 15 by non-radioactive electromagnetic waves of mm range and low intensity from generator 16, after that the biomaterial which have been obtained from discharge from block of centrifuges for isolating photosynthesizing microorganisms (algae, bacteria) pigment 12 enters packaging block 17.

Photochemical properties of pigment (bacteriorhodopsin) films can be varied (for special consumers) in bioreactor for bacteria cultivation, microorganism stress bioreactor 9 and in block of output control 15 with the help of the following methods:

a) by changing natural chromophore for a modified one;
b) by physical and chemical actions, e.g., non-radioactive electromagnetic radiation of mm range and low intensity from generators which have regulated parameters of radiation (wavelength, spectral density, power, period of irradiation, irradiation regime: pulse, continuous, modulated).
c) by point replacements of definite aminoacid residues using methods of genetic engineering [46-58].

Intermediate product (discharge from block of centrifuges for isolating photosynthesizing microorganisms (algae, bacteria) pigment) can be further used as growth activator for biological objects of different kinds (microorganisms, plants, animals), as a producer (initial materials) in foodstuff, pharmaceutical, cosmetic industries and for active sludge growing in purification installations.

End product (pigment, bacteriorhodopsin) can be further used in nano-electronics, optics, medicine.

The invention proposed permits to develop a technologically simple, environmentally friendly and safe method for increasing photosynthesizing microorganisms (algae, bacteria) and pigment (bacteriorhodopsin) biomass.

Biological effect of applying non-radioactive electromagnetic radiation of mm range and low intensity to photosynthesizing microorganisms is manifested in increase of their biomass which is obtained during cultivation, as well as in increase of pigment content in their cells. Experimental data which are stated in examples 1, 2, 3 are taken from [53, 54].

### Example N1

Cultivation of microorganisms, in particular culture Haematococcus pluvialis, is known to have at least two phases:

production of microorganisms biomass;

production of pigment biomass, e.g. pigment Astaxanthin.

Known technologies for increasing microalgae biomass output such as changes in illumination. additional enrichment of cultivated medium with carbon dioxide gas and other nutrients, give insignificant biomass increase (from 7 to 12%) [25, 28, 59-65].

Laboratory experiments on microalgae irradiation by electromagnetic waves of mm range have shown significant biomass increase (for 250-410%). that's why electromagnetic radiation in mm range has been chosen as outer physical factor with electromagnetic radiation of microalgae being carried out at each stage of biotechnological process.

Experiments have been carried out by extracting samples of solutions directly from devices of an Israeli industrial installation (tubular bioreactor) for microalgae cultivation in the phase of producing biomass of microalgae culture Haematococcus pluvialis, in the phase of microorganism stress and in the phase of obtaining pigment Astaxanthin. Solutions which have been taken at different points of biotechnological process were installed in shaker flasks at test beds for irradiation, then after irradiation each solution (in flasks) went through cultivation under laboratory conditions (according to subsequent operations and phases of the biotechnological process). This kind of research is usually called semi-industrial modeling. Microalgae culture Haematococcus pluvialis, physiological solution for cultivation and water used for physiological solution preparation served as objects of irradiation (as research materials) in this semi-industrial experiment. Chemical composition of cultivated medium for culture Haematococcus pluvialis was a standard one (according to technology of microalgae cultivation used).

Temperature regime for microalgae cultivation has been in August 2003 within the limits: in the daytime—+(32-38)° C. and at night—+(24-27)° C.; in December 2003 the temperature regime was within the limits: in the daytime—+(28-30)° C. and at night—+(20-23)° C. Correspondingly, illumination of bioreactor pipes and shaker flasks was equal in August 2003 to 1800-2500 lux, and in December 2003 it was equal to 1100-1700 lux.

Increase in microalgae biomass and pigment has been determined by nephelometric method.

Installations used in semi-industrial experiment are represented by low-power industrial generators of electromagnetic waves of mm range as well as serially produced equipment for chemical-biological analysis of cultivated microalgae solutions.

Radiation frequency of generators for non-radioactive radiation of electromagnetic waves of mm range and low intensity are the same as frequencies of non-transparent windows for electromagnetic waves propagation in atmosphere within 40-75 GHz. Radiation intensity at each generation frequency was not more than 1-3 mcWatt per square cm of irradiated surface. Radiation spectrum was a noise one with radiation band within 300-700 MHz. Period of each flask irradiation was equal to 30 min.

Shake flasks with cultivated medium (with microalgae) or flasks with physiological solution for cultivation were irradiated by electromagnetic waves of mm range and low intensity. Cultivated medium with microalgae Haematococcus pluvialis was extracted from bioreactor of continuous biotechnological process simultaneously in phases of microalgae cultivation (phase D), stress development (phase S) and pigment Astaxanthin production (phase P). Physiological solution has been extracted from preparation tank. Both the cultivated medium with microalgae and physiological solution were placed in flasks which were irradiated either externally or internally at each of the frequencies generated by electromagnetic radiation of mm range and low intensity.

Semi-industrial experiments on irradiating microalgae culture of Haematococcus pluvialis by electromagnetic waves of millimeter range and low intensity have been carried out in Israel in August-December 2003 at an industrial tubular bioreactor in two stages.

The first stage of semi-industrial tests provides for conducting experiments on irradiating cultivated medium (microalgae). The peculiarity of the first stage is that irradiation of each microalgae solution has been carried out separately: irradiation of cultivated medium before operation on biomass cultivation (phase D); then irradiating cultivated medium after biomass cultivation (before carrying out stress operation) (phase S); and then irradiation of cultivated medium which has undergone two operations—biomass cultivation and stress operation (before operation on additional production of pigment Astaxanthin) (phase P).

The results of this first stage are given in tables 1, 2, 3. The tables use the following designations: letters D, S, P designate the phase of biotechnological process, the subsequent letters and numbers stand for:
Control (C)—control group of algae in quartz flask (without irradiation)
1G1—a group of algae which was irradiated by electromagnetic waves at frequency f1 outside the quartz flask,
1G2—the same but with irradiation inside the flask,
2G1—a group of algae which was irradiated by electromagnetic waves at frequency f2 outside the quartz flask,
2G2—the same but with irradiation inside the flask,
3G1—a group of algae which was irradiated by electromagnetic waves at frequency f3 outside the quartz flask,
3G2—the same but with irradiation inside the flask.

TABLE 1
Current values of algae biomass Haematococcus pluvialis
during cultivation phase D

| | 1st day | 3rd day | 5^{th} day | 7th day | 9th day |
|---|---|---|---|---|---|
| | D.W. | D.W. | D.W. | D.W. | D.W. |
| | g/l | g/l | g/l | g/l | g/l |
| DC | 0.46 | 0.66 | 1.03 | 1.47 | 2.00 |
| D1G1 | 0.46 | 0.66 | 1.09 | 1.60 | 2.14 |
| D1G2 | 0.46 | 0.71 | 1.15 | 1.76 | 2.39 |
| D2G1 | 0.46 | 0.64 | 1.12 | 1.44 | 1.91 |
| D2G2 | 0.46 | 0.67 | 1.17 | 1.70 | 2.20 |
| D3G1 | 0.46 | 0.66 | 1.06 | 1.51 | 2.03 |
| D3G2 | 0.46 | 0.67 | 1.07 | 1.59 | 2.11 |

TABLE 2
Current values of algae biomass Haematococcus pluvialis
and pigment Astaxanthin during stress phase S

| | 1st day | | 3rd day | | 5th day | | 7^{th} day | | 9th day | |
|---|---|---|---|---|---|---|---|---|---|---|
| | D.W. | | D.W. | | D.W. | Asta | D.W. | Asta | D.W. | Asta |
| | g/l | Asta mg/l | g/l | Asta mg/l | g/l | mg/l | g/l | mg/l | g/l | mg/l |
| SC | 0.45 | 2.71 | 1.49 | 23.69 | 2.25 | 47.65 | 2.12 | 48.87 | 2.48 | 51.42 |
| S1G1 | 0.45 | 2.71 | 1.53 | 23.48 | 2.53 | 60.32 | 2.93 | 92.77 | 3.41 | 112.72 |
| S1G2 | 0.45 | 2.71 | 1.49 | 25.91 | 2.06 | 45.68 | 2.17 | 54.73 | 2.66 | 105.65 |
| S2G1 | 0.45 | 2.71 | 1.51 | 28.38 | 2.19 | 51.18 | 2.13 | 46.09 | 2.03 | 42.74 |
| S2G2 | 0.45 | 2.71 | 1.58 | 27.54 | 2.28 | 52.72 | 2.20 | 55.54 | 2.34 | 52.43 |
| S3G1 | 0.45 | 2.71 | 1.46 | 27.43 | 2.21 | 50.69 | 2.01 | 49.52 | 2.79 | 52.65 |
| S3G2 | 0.45 | 2.71 | 1.46 | 24.77 | 2.51 | 63.66 | 3.00 | 89.40 | 3.40 | 109.03 |

TABLE 3
Current values of algae biomass Haematococcus pluvialis
and pigment biomass during the phase of pigment production P

| | | |
|---|---|---|
| 1st day | 5the day | 7the day |

| | D.W. | Asta | D.W. | Asta | D.W. | Asta |
|---|---|---|---|---|---|---|
| | g/l | mg/l | g/l | mg/l | g/l | mg/l |
| PC | 1.82 | 18.60 | 4.40 | 114.37 | 5.27 | 139.16 |
| P1G1 | 1.82 | 18.60 | 4.14 | 106.11 | 5.22 | 127.98 |
| P1G2 | 1.82 | 18.60 | 3.81 | 89.40 | 4.80 | 106.27 |
| P2G1 | 1.82 | 18.60 | 3.88 | 106.76 | 4.93 | 125.87 |
| P2G2 | 1.82 | 18.60 | 3.88 | 97.52 | 4.91 | 111.62 |
| P3G1 | 1.82 | 18.60 | 3.50 | 86.81 | 4.70 | 118.58 |
| P3G2 | 1.82 | 18.60 | 3.42 | 82.05 | 4.51 | 104.65 |

The second stage of semi-industrial tests provides for carrying out experiments on irradiating the physiological solution (before mixing it with algae). Peculiarity of the second stage is that physiological solution (after being irradiated) is used for diluting the cultivated medium before operation on algae cultivation (phase D) and for diluting the cultivated medium before stress operation (phase S) at the stage of producing pigment Astaxanthin.

The results of the second stage are given in Tables 4, 5, 6, 7, 8, 9.

TABLE 4
Current values of algae biomass when using irradiated
physiological solution during cultivation phase D

| | Control | DIG2 |
|---|---|---|
| Days | (g/l) | (g/l) |
| 1st day | 0.34 | 0.34 |
| 3rd day | 0.60 | 0.56 |
| 5th day | 0.85 | 1.04 |
| 7th day | 1.36 | 2.00 |
| 9th day | 1.44 | 2.47 |
| 11th day | 1.56 | 2.92 |

TABLE 5
Current values of algae biomass when using
irradiated cultivated medium during cultivation phase D

| | Control | DIG2 |
|---|---|---|
| Days | (g/l) | (g/l) |
| 1st day | 0.50 | 0.50 |
| 3rd day | 0.80 | 0.70 |
| 5th day | 0.91 | 1.08 |
| 7th day | 1.40 | 1.73 |
| 9th day | 1.97 | 2.38 |

TABLE 6
Current values of algae biomass when using
irradiated physiological solution during stress phase S

| | Control | DIG2 |
|---|---|---|
| Days | (g/l) | (g/l) |
| 1st day | 0.50 | 0.50 |
| 3rd day | 1.08 | 1.05 |
| 5th day | 1.75 | 1.84 |
| 7th day | 1.96 | 2.18 |
| 9th day | 2.24 | 2.38 |
| 11th day | 2.26 | 2.47 |

TABLE 7
Current values of algae biomass when using
irradiated cultivated medium during stress phase S

| | Control | DIG2 |
|---|---|---|
| Days | (g/l) | (g/l) |
| 1st day | 0.50 | 0.50 |
| 3rd day | 2.11 | 2.06 |
| 5th day | 2.15 | 2.54 |
| 7th day | 2.18 | 3.04 |
| 8th day | 2.41 | 3.37 |

TABLE 8
Current values of pigment biomass when using irradiated
cultivated medium during stress phase S

| | Control | DIG2 |
|---|---|---|
| Days | (mg/l) | (mg/l) |
| 1st day | 2.00 | 2.00 |
| 3rd day | 46.00 | 64.00 |
| 5th day | 52.00 | 96.00 |
| 7th day | 52.58 | 113.00 |

TABLE 9
Current values of pigment biomass when using irradiated
physiological solution during stress phase S

| | Control | DIG2 |
|---|---|---|
| Days | (mg/l) | (mg/l) |
| 1st day | 2.00 | 2.00 |
| 3rd day | 18.00 | 16.00 |
| 5th day | 41.00 | 38.00 |
| 7th day | 56.18 | 48.64 |
| 8th day | 62.41 | 58.37 |
| 10th day | 81.56 | 78.92 |

Increase in microalgae biomass is observed in phases D, S during the industrial process of algae cultivation. As follows from tables 1 and 2 weight of microalgae biomass Haematococcus pluvialis which has been irradiated before phases D and S attained correspondingly 119% and 141% of algae biomass control group. Data of table 2 show that the weight of isolated (produced) pigment Astaxanthin was equal to 187% (on the 7th day of cultivation) and 219% (on the 9th day of cultivation) of the weight of pigment isolated from microalgae control group.

Tables 4 and 5 show that in case the physiological solution was irradiated before phase D and then added to culture medium with microalgae, biomass of such microalgae group increases with further cultivation (in phase D) for 150-187% in comparison to the control biomass of microalgae group which wasn't subjected to radiation. Biomass of such cultivated microalgae group increases for 1.55 times in comparison to microalgae group which was completely irradiated before phase D.

Absence of non-specific initial conditions influence on cultivation process shows stable manifestation of biological effects with microwave irradiation (which was represented in tables 1, 2, 4, 5, 6, 7, 8,) which was certified by experimental data obtained during an experiment using the same equipment of industrial tubular bioreactor in August and September 2003 (at different meteorological conditions). Low intensity of radiation (microwatt) and short duration of radiation (short time of radiation exposure) (up to 30 minutes) makes it possible to state that vigorous growth of microalgae and pigment biomass is caused not by temperature increase but only by the influence of electromagnetic radiation on cultivated medium.

The results of semi-industrial experiments conducted in Israel completely certified biological effects of waves in mm range which have been obtained in Russia and Ukraine during laboratory tests. These tests and semi-industrial experiments carried out for the first time, have shown that irradiation by electromagnetic waves causes intensive development in cultures of photosynthesizing microalgae and their pigments.

### Example No 2

Value of photosynthetic activity of radiated and control algae
in the process of growth (biomass, g/l)

| Culture age | Radiation time | Culture kinds | |
|---|---|---|---|
| (days) | (min) | Spirulina platensis | Platimonas viridis |
| 10 | 30 | 0.25 | 0.19 |
| 10 | 60 | 0.12 | 0.25 |
| 10 | 120 | 0.10 | 0.20 |
| 10 | 360 | 0.04 | — |
| 10 | Control | 0.07 | 0.20 |
| 20 | 15 | 0.60 | 0.61 |
| 20 | 30 | 1.05 | 0.61 |
| 20 | 60 | 0.82 | 0.70 |
| 20 | 120 | 0.52 | 0.47 |
| 20 | 360 | 0.22 | — |
| 20 | Control | 0.46 | 0.42 |
| 30 | 15 | 2.52 | 1.00 |
| 30 | 30 | 4.25 | 1.10 |
| 30 | 60 | 0.81 | 1.20 |
| 30 | 120 | 0.52 | 0.72 |
| 30 | 360 | 0.22 | — |
| 30 | Control | 2.05 | 0.68 |

Value of photosynthetic activity of radiated and non-radiated algae in
the process of growth (biomass, g/l)

| | | Culture kinds | | | |
|---|---|---|---|---|---|
| | | Spirulina platensis | | Platimonas viridis | |
| Culture age | Radiation time | non- | | non- | |
| (days) | (min) | radiated | radiated | radiated | radiated |
| 10 | 15 | 0.15 | 0.30 | 0.05 | 0.16 |
| 10 | 30 | 0.20 | 0.23 | 0.05 | 0.18 |
| 10 | 60 | 0.13 | 0.13 | 0.05 | 0.15 |
| 10 | 120 | 0.12 | 0.10 | 0.12 | 0.16 |
| 10 | 360 | 0.07 | 0.43 | 0.20 | 0.16 |
| 20 | 15 | 0.75 | 1.40 | 0.31 | 0.60 |
| 20 | 30 | 0.71 | 1.07 | 0.31 | 0.47 |
| 20 | 60 | 0.67 | 1.05 | 0.31 | 0.54 |
| 20 | 120 | 1.52 | 1.03 | 0.27 | 0.28 |
| 20 | 360 | 0.32 | 0.26 | 0.40 | 0.40 |
| 30 | 15 | 2.40 | 5.20 | 0.40 | 0.91 |
| 30 | 30 | 2.12 | 4.16 | 0.40 | 0.92 |
| 30 | 60 | 1.96 | 2.08 | 0.40 | 1.15 |
| 30 | 120 | 3.24 | 2.92 | 0.44 | 0.49 |
| 30 | 360 | 1.56 | 1.44 | 0.57 | 0.52 |

### Example No 3

Resonance effect with radiation of Spirulina culture by
electromagnetic radiation of mm range

| Experiment variants | | | | | |
|---|---|---|---|---|---|
| Wavelength, | power, | Culture age (days) | | | |
| (mm) | (mcWatt/cm²) | 10 | 20 | 30 | pH |
| 6.95 | 2.43 | 0.11 | 0.40 | 0.64 | 9.86 |
| 7.00 | 1.75 | 0.21 | 0.78 | 1.20 | 9.93 |
| 7.05 | 0.80 | 0.26 | 0.95 | 1.50 | 9.97 |
| 7.10 | 1.40 | 0.24 | 0.85 | 1.20 | 10.03 |
| 7.15 | 2.17 | 0.24 | 0.75 | 1.40 | 9.98 |
| 7.20 | 2.19 | 0.24 | 0.95 | 1.55 | 9.97 |
| 7.25 | 2.16 | 0.20 | 0.75 | 1.20 | 9.95 |

The analysis of data an example 3 shows, that depending on frequency of an irradiation the size of an increment of a biomass of algae is various at their identical stages of cultivation. Thus, at a stage of 10-20 days the greatest gain of a biomass at an irradiation on length of a wave of 7.20 mm, and at a stage of 20-30 days the greatest gain of a biomass at an irradiation on length of a wave of 7.15 mm.

The aim of optimizing microorganism cultivation process is to provide a stable increase in the mass of biosynthesis output products for several times and a decrease in expenditures for microorganism biosynthesis including microorganisms and their pigments taking into account given environmental and physiological conditions of cultivation [66, 71-76].

Optimization of microorganism cultivation process is based on population approach to obtaining the microorganism biomass and products of its life activity and includes elements of theory (rules) of regulating biotechnological processes. At present cultivation process optimization is completely equipped with devices: there exist different types of bioreactors, power measuring equipment, regulation and ingredients feeding devices. That's why adjustment of regulation devices for maintaining optimum structure of microalgae population makes it possible to carry out biosynthesis of the output (end) product with maximum effectiveness—with biomass increase for several times. Adjustment is performed in such a way that microorganisms cultivation (bacteria, yeasts) with constant growth rate of microorganisms number in bioreactor takes place in three regimes of different growth of microorganisms number as the function of ratio of carbon to nitrogen C:N in cultivated medium: a) regime definitely limited in relation to carbon, but with the excess of nitrogen, b) transient regime (limited in relation to both nutrients) where both carbon and nitrogen quantities were lower than the limit, and c) regime definitely limited in relation to nitrogen but with excess of carbon.

Microorganisms growth in industrial processes is always in one way or another controlled by the limited quantity of nutrients, and this limitation in relation to definite substances is often used for forcing microbe cultures into entering the physiological stage of producing the end product.

Optimization of biotechnological process is based on functioning of two biological models of cultivation and microorganisms stress, halobacteria in particular, and on the operation of situational algorithm in decision block which permits to choose sequence of actions with equipment for proposed device control parameters regulation, e.g. with control schemes of block for raw materials supply 6 and bioreactor of bacteria cultivation 5.

Optimization of biotechnological processes on the basis of dynamic evaluation of microorganism nutrient needs permits to obtain the following results:

decrease in raw material expenditures;

increase in effectiveness for several times in the process of products growth and biosynthesis in media prepared on the basis of complex organic raw material substrates;

increase in product output due to optimum values of limiting factors in nutrient media.

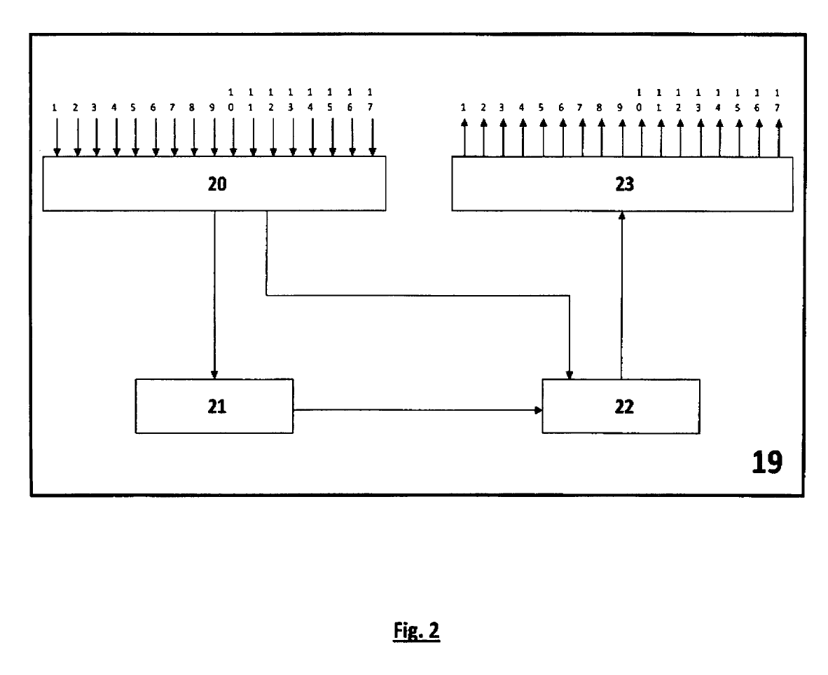

Optimization of the processes of photosynthesizing microorganisms (algae, bacteria) cultivation and their pigments production is carried out by means of control block 19 (FIG. 2). Register 20 of analog-to-digital converter in control block 19 gets current values of controlled parameters from water treatment block 2, block of raw material supply 6, bioreactors 5 and 9 of bacteria cultivation and microorganism stress, bidistilled water source 11, separator for photosynthesizing microorganisms (algae, bacteria) selection 4, separator for photosynthesizing microorganisms (algae, bacteria) sorting 8, block of centrifuges 12, drying block 13, output control block 15, packaging block 17, generators of non-radioactive radiation of mm range and low intensity 3, 7, 10, 14, 16. Current values of controlled parameters from devices go from register 20 of analog-to-digital converter to inputs of digital (analog) elements of model 21 for processes of photosynthesizing microorganisms (algae, bacteria) cultivation and stress and the output data from this model are transmitted to decision block 22 which transmits control signals for regulating each control parameter in all the elements of the proposed device. These control signals enter register 23 of analog-to-digital converter in control block 19. Register 23 of analog-to-digital converter in control block 19 transmits signals to control outputs of control schemes in water treatment block 2, block of raw material supply 6, bioreactors 5 and 9 of bacteria cultivation and microorganism stress, separator for photosynthesizing microorganisms (algae, bacteria) selection 4, separator for photosynthesizing microorganisms (algae, bacteria) sorting 8, block of centrifuges 12, drying block 13, output control block 15, packaging block 17, generators of non-radioactive radiation of mm range and low intensity 3, 7, 10, 14, 16 and source of bidistilled water 11.

The proposed solution of the installation structure corresponds in its basic part to the logics method proposed, that's why it is considered that a more detailed description of the device mentioned is not necessary within the framework of the given application for the invention [77-92].

It is important, therefore, that the scope of the invention is not construed as being limited by the illustrative embodiments set forth herein. Other variations are possible within the scope of the present invention as defined in the appended claims. Other combinations and sub-combinations of features, functions, elements and/or properties may be claimed through amendment of the present claims or presentation of new claims in this or a related application. Such amended or new claims, whether they are directed to different combinations or directed to the same combinations, whether different, broader, narrower or equal in scope to the original claims, are also regarded as included within the subject matter of the present description.

### LITERATURE

1. Handbook of microalgae mass culture (1986), Ed: A. Richmond, Boca Raton (Flo), 528.
2. Bickel-Sandkotter Susanne, Gartner Wolfgang, Dane Michaela Conversion of energy in halobacteria: ATP synthesis and phototaxis/Bickel-Sandkotter Susanne, Gartner Wolfgang, Dane Michaela//Arch. Microbiol.-1996.-166, No 1, 1-11.
3. I. Lang (1996), Cultivation of marine unicellular algae, Lowestoft, p. 31. Utilization of D-leucine by Halobacterium halobium VO107/Tanaka Mikiei, Mukohata Yasuo, Yuasa Seiji II Can. J. Microbiol.-1996.-42, No 9, 973-976.
4. Fei Xiu-Geng, Lu Shan, Bao Ying (1998), Algae cultivation: A new applied field for biotechnology//Chinese journal of Oceanology and Limnology, v. 16, s. 1, 158-161.
5. Stephanus V. Mandagi and Ian White (2005), A new technique for algae cultivation to minimize impacts on tropical, coastal environments, Centre for Research Australian National University, Can berra, ACT 0200,83.
6. Shpigel, M., Neori, A. (2007). Microalgae, macroalgae, and bivalves as biofilters in land-based mariculture in Israel. In: Bert, T. M. (Ed.), Ecological and genetic implications of aquaculture activities. (Methods and technologies in fish biology and fisheries. 6). Springer, 433-446
7. Ask, E. I., & Azanza, R. V. (2002). Advance in cultivation technology of commercial eucheumatoid species: a review with suggestions for future research. Aquaculture, 206(34), 257-277.
8. Olafson, E., Johnston, R. W., & Simon, G. M. N. (1995). Effect of intensive algae in a tropical lagoon//Journal of Experimental Marine Biology & Ecology, 191, (1), 111-117.
9. U.S. Pat. No. 5,888,791 (1999) <<Method of producing bacteriorhodopsin and carotenoids by electrostatic treatment of Halobacterium halobium” (1999).
10. U.S. Pat. No. 5,882,849 (1999) <<Method of control of Haematococcus spp. Growth process>>.
11. U.S. Pat. No. 5,541,056 (1996) <<Method of control of microorganism growth process>>.
12. U.S. Pat. No. 6,805,800 (2004) “Method for recovering pigments from algae cultures”.
13. U.S. Pat. No. 6,858,430 (2005) “Process for cultivation of algae”.
14. RU patent No. 2 148 903 (2000) “Biological Object Vitality Improving Method”.
15. U.S. Pat. No. 4,868,123 (1989) “Apparatus for the intensive, controlled production of microorganisms by photosynthesis”.
16. U.S. Pat. No. 5,998,781 (1997) “Apparatus for millimeter-wave signal generation”.
17. U.S. Pat. No. 5,982,245 (1999) “Radiating oscillator apparatus for micro- and millimeter waves”.
18. U.S. Pat. No. 5,641,650 (1997) “Expression of heterologous polypeptides in halobacteria”
19. RU Patent No. 2 228 352 (2003) “Method of producing biomass and products of its vital activities”.
20. U.S. Pat. No. 4,952,511 (1990)) “Photobioreactor”.
21. U.S. Pat. No. 5,958,761 (1996) “Bioreactor and system productivity of photosynthetic algae”.
22. U.S. Pat. No. 4,676,956 (1987) “Apparatus for photosynthesis”.
23. U.S. Pat. No. 5,614,378 (1997) “Photobioreactors and closed support systems and artificial lungs containing”.
24. Patent WO 1991/005849 “The method of cultivation of photosynthetic microorganisms and cells of photosynthetic macrophytes”
25. U.S. Pat. No. 4,554,390 (1985) “Method for harvesting algae”
26. U.S. Pat. No. 4,676,956 (1987) “Apparatus for photosynthesis”.
27. U.S. Pat. No. 7,144,733 (2006) “Bio-synthetic photostimulators and methods of use”
28. Patent WO97/28274, Boussiba S., Vonshak A., Cohen Z., Richmond A. (1997) “A procedure for large scale production of Astaxanthin from Haematococcus”
29. U.S. Pat. No. 7,080,478 (2006) “Technology for cultivation of Porphyra and other algae in land-based sea water ponds”.
30. U.S. Pat. No. 5,888,791 (1991) “Method of producing bacteriorhodopsin and carotenoids by electrostatic treatment of Halobacterium halobium”.
31. Frohlich H. (1973), Collective behaviour of non-linearly couple oscillating fields. With applications to biological systems/Collective Phenomena., Vol. 1. P. 101-109.
32. Grundler W. (1983), Non-thermal resonant effects of microwaves on the growth of yeast cultures, W. Grundler et al, In: “Coherent excitation in biological systems”, p. 21-37.
33. Presman A. S. (1968), Electromagnetic fields and wildlife., Moscow, Nauka, 28 p.
34. Grundler W., Kaiser F., Keilmann F., Walleczek J., (1992): Mechanisms of electromagnetic interaction with cellular systems//Naturwissenschaften 79:551-5597.
35. Furia L., Hill D. W., Gandhi O. P. (1986), Effect of millimeter-wave irradiation on growth of Saccharamyces cerevisiae//IEEE Trans. Biomed. Eng, BME-33, 11 :. 993-999.
36. Gandhi O. P. (1983), Some basic properties of biological tissues for potential biomedical applications of millimeter waves, J. Microwave Power, Vol. 18, 3 :95-304.
37. Gos P., Eicher B., Kohli J., Heyer W.-D. (1997), Extremely high frequency electromagnetic fields at low power density do not affect the division of exponential phase Saccharomyces cerevisiae cells, Bioelectromagnetics., Vol. 18, 1: 142-155.
38. Grundler W., Keilmann F. (1978), Nonthermal effects of millimeter microwaves on yeast growth, Z. Naturforsch., Vol. 33, 1: 15-21.
39. Grundler W., Keilmann F. (1989), Resonant microwave effect on locally fixed yeast microcolonies, Z. Naturforsch, Vol. 44, 5: 863-866.
40. Grundler W., Keilmann F., Frohlich H. (1977), Resonant growth rate response of yeast cells irradiated by weak microwaves, Physiol. Lett., Vol. 62A, 3 : 463-466.
41. M. Blank, (1995), Biological effects of environmental electromagnetic fields: Molecular mechanisms, Biosystems 35: 2-3
42. Khizhnyak E. P. and Ziskin M. C., (1996), Temperature Oscillations in Liquid Media Caused by Continuous (Non-modulated) Millimeter Wavelength Electromagnetic Irradiation, Bioelectromagnetics, v. 17, pp. 223-229.
43. Nishizuka Y. (1992), Intracellular signalling by hydrolysis of phospholipids and activation of protein kinase C, Science, Vol. 258, 3: 607-614
44. Tambiev A. X., Gusev M. V., Kirikova N. N., Betskii O. V. (1986): Stimulation of Growth of Cyanobacteria by Millimeter electromagnetic Radiation of Low Intensity, X1V Inter. Congress of Microbiology, England
45. Slavin V. E., (2000): Phenomenon of radioelectronic resonance and its application in Hi-Tech technologies, Proceedings of the IASA, Israel, Issue N 1: 30-31.
46. Gulyayev Yu. V., Vainer G. B., Gubanova Yu. K. et. al., Changes of energy and phospholipin metabolism in E. coli cells under millimeter UHF-range influence.//Report digest. Application of low-intensity millimeter radiation in biology and medicine.—M.: IRE AN USSR, 1986.
47. Golant M. B., Bryuhkova A. K., Rebrova T. B. Some regularities of millimeter range electromagnetic radiation influence on microorganisms./Report digest. Application of low-intensity millimeter radiation in biology and medicine.—M.: IRE AN USSR, 1985.
48. Devyatkov N. D., Golant M. B., Rebrova T. B. About mechanism of low-intensity millimeter range electromagnetic radiation influence on living organisms and prospects of its application in connection to this.//Reports thes. of All-Union symp. “Biological effect of electromagnetic fields”.—Pushino: ONTI NTsBI AN USSR, 1982.
49. Devyatkov N. D., Chemov Z. S., Betskiy O. V., Putvinskiy A. V. Millimeter radiation influence on biological membranes.//Reports thes. of All-Union symp. “Biological effect of electromagnetic fields”.—Pushino: ONTI NTsBI AN USSR, 1982.
50. Andreev V. S., Pechorina T. A. Non-thermal intensity EHF-radiation influence heredity of microorganisms.//Reports thes. of Int. symp. “Non-thermal intensity millimeter waves in medicine”.—M.: IRE AN USSR, 1991.
51. Shub G. M., Luneva I. O., Denisova S. G., Ostrovskiy N. V. Mm-wave influence on bacteria in experiments in vitro and in vivo.//Report digest. Millimeter waves in medicine and biology.—M.: IRE AN USSR, 1995.
52. Kholodnaya L. S., Pozur V. K., Lyubchenko T. A. EHF-radiation influence on immunological properties of conditionally pathogenic bacteria. II Report digest. Millimeter waves in medicine and biology.—M.: IRE AN USSR, 1995.
53. Tambiyev A. Kh., Kirikova N. N., Lapshin O. M. et al. Effect of millimeter and centimeter electromagnetic radiation combined influence on microalgae productivity.//Report digest. Millimeter waves in medicine and biology.—M.: IRE AN USSR, 1989.
54. Rai S., Singh S. P., Samarketu et al. Effect of modulated microwave frequencies on the physiology of a cyanobacterium Anabaena doliolum.—Electro- and Magnetobiology, 1999, 18 (3).
55. U.S. Pat. No. 5,876,767 (1999) Apparatus for layerwise production on object using laser sintering”.
56. Nox P. P., Pashchenko V. Z., Logunov S. L. et al. EMR EHF influence on dynamics of triplet states formation in photosynthesizing reaction centers of purple algae and on RKR spectrum of carotinoid component.//Reports thes. of Int. symp. “Non-thermal intensity millimeter waves in medicine”.—M.: IRE AN USSR, 1991.
57. Lukashev Ye. P., Kononenko A. A., Rubin A. B. Influence of EMR EHF on electron and proton transfer in light-sensitive natural chromophore-protein complexes. II Reports thes. of Int. symp. “Non-thermal intensity millimeter waves in medicine”.—M.: IRE AN USSR, 1991.
58. Motzkin Sh. Low power continuous wave millimeter irradiation falls to produce biological effects in lipid vesicles. Mammalian muscle cells, and E. coli. II Reports thes. of Int. symp. “Non-thermal intensity millimeter waves in medicine”.—M.: IRE AN USSR, 1991.
59. Boussiba S., Fan L., Vonshak A. Enhancement and determination of Astaxanthin accumulation in green alga Haematococcus pluvialis.//Methods in enzomology.-1993.—No 213.—P. 386-392.
60. Kobayashi M., T. Kakizono, K. Yamaguchi, N. Nishio and S. Nagai. Growth and Astaxanthin formation of Haematococcus pluvialis in heterotrophic and mixotrophic conditions.//Journal of fermentation and Bioengineering.-1992.—No 74.—P. 61-63.
61. Biological aspects of low intensity millimeter waves/N. D. Devyatkov, O. V. Betskii (Eds).—Moscow: Seven Plus, 1994-324 p.
62. Forman P. Atoms in electromagnetic fields//Science.-1995.—Vol. 268.—No 5214.—P. 1212-1214.
63. Richmond A. Optimal orientation and inclination of photobioreactors for growing photoautotrophic microorganisms outdoors. II Appl. Biochem. & Biotechnol.-1997—Vol. 63.—No 5.—P. 649-659.
64. Betskii O., Tambiev A., Kirikova N., Lebedeva N., Slavin V. Low intensity millimeter waves and their application in Hi-Tech Technologies. II Scientific Israel—Technological Advantages.-2000.—No 2.—P. 97-109.
65. Velizarov S., Raskmark P., Kwee S. The effects of radiofrequency fields on cell proliferation are non-thermal.//Bioelectrochem Bioenerg.—1999.—No 48(1). P. 177-180.
66. Egli T., Zinn M. The concept of multi-nutrient-limited growth of microorganisms and its application in biotechnological processes. M Biotechnol. Adv. 2003. N 22, p. 3543.
67. Severina I. S. Soluble guanilcicleasa in the molecular mechanism of physiological effects of nitric oxide//Biochemistry, 1998, V. 63. N 7. P. 939-997.
68. Furchgott R. F., Jothiandan D. Endothelium-dependent and independent vasodilation involving cyclic GMP: relaxation induced by nitric oxide, carbon monoxide and light//Blood Vessels. 1991. V. 28. P. 52-61.
69. Ignarro L. J. Biosynthesis and metabolism of endothelium-derived nitric oxide//Annu. Rev. Pharmacol. Toxicol. 1990. V. 30. P. 535-560.
70. Palmer R. M., Ferige A. G., Moncada S. Nitric oxide release accounts for the biological activity of endothelium-derived relaxing factor//Nature. 1987. V327. P. 524-526.
71. Marxen et al, (2003) “A photobioreactor system for computer controlled cultivation of microalgae, Journal of Applied Psychology, v. 17, N 6, pp. 535-549.
72. Gretzova N. V., Shein A. G. (2006), Modeling of electromagnetic radiation influence on biological objects. Biomedical technologies and radioelectronics, No 4, p. 18-35.
73. Bulgakov N. G., Levich A. P. The nitrogen Phosphorus ratio as a factor regulating phytoplankton community structure//Archiv fir Hydrobiologie. 1999. V. 146. No. 1. Pp. 3-22.
74. Levich A. P., Maximov V. N., Bulgakov N. G. Theoretical and experimental ecology of phytoplankton: managing the structure and functions of communities. Moscow, NIL Printing House, 1997, 192 p.
75. Levich A. P. Optimization modelling of multi-component open systems under conditions of competitiveness for multiple resources. Thesis of the 5th International Conference “Mathematics. computer, education”. Moscow, 1998, p. 118.
76. Levich A. P. Variational modelling theorems and algocoenosis functioning principles I Ecological Modelling. 2000. V. 131. No. 2-3. Pp. 207-227.
77. Holmes, M. L., et al., “A Plasmid Vector with a Selectable Marker for Halophillic Archaebacteria”, Journal of Bacteriology, 172:756-761 (1990).
78. Ni, B., et al., “An efficient system for the synthesis of bacteriorhodopsin in Halobacterium halobium”, Gene, 90:169-172 (1990).
79. Baofu Ni, Man Chang, A. Duschl, J. Lanyi, R. Needleman, Gene 90 (1990), p. 169-172, “An efficient system for the synthesis of bacteriorhodopsin in Halobacterium halobium”.
80. N. Hampp, C. Brauchle, D. Oesterhelt, SPIE, vol. 1125, Thin Films in Optics (1989), Optical Properties of Polymeric Films of Bacteriorhodopsin and its Functional Variants New Materials for Optical Information Processing.
81. Studies in Organic Chemistry 40—PHOTOCHRO H. Durr Elsevier, Amsterdam NL, Chapter 29, Bacteriorhodopsin and its functional variants, N. Hampp, p. 954-975.
82. Blaseio, U., et al., “Transformation of Halobacterium halobium: Development of vectors and investigation of gas vesicle synthesis”, Proc. Natl. Acad. Sci. USA, 87:6772-6776 (1990).
83. Birge, R. R., “Photophysics and Molecular Electronics Applications of the Rhodopsins”, Annu. Rev. Phys. Chem., 41:683-733 (1990).
84. Betlach, M., et al., “Characterization of a halobacterial gene affecting bacteriorhodopsin gene expression”, Nucleic Acids Research, 12:7949-7959 (1984).
85. Dunn, R., et al., “The bacteriorhodopsin gene”, Proc. Natl. Acad. Sci. USA, 78:6744-6748 (1981).
86. Dunn et al., “Structure-Function Studies on Bacteriorhodopsin,” J. Biol. Chem., 262(19):9246-9254 (1987).
87. Krebs et al., “Expression of the Bacteriorhodopsin gene in Halobacterium halobium Using a Multicopy Plasmid,” PNAS USA, 88:859-863 (1991).
88. Encyclopaedia of Lasers and Optical Technology by R. Heyers, Ed. Academic Press, Inc., 1991, pp. 397-400.
89. Quarterly Reviews of Biophysics entitled “Bacteriorhodopsin: a biological material for information processing”, 24, 4 (1991), pp. 425-478.
90. “Biopolymers for Real-Time Optical Processing”, Bazhenov et al. pp. 103-144 (1989). McDowell et al. “Transglutaminase Modification of Rhodopsin in Retinal Rod Outer Segment Dick Membranes” Arch. Biochem. Biophys. (1986) 249(2): 506-514.
91. Gaffney, B. J., “Chemical and biochemical cross-linking of membrane components”, 1985, Biochimica Biophysica Acta, vol. 822, pp. 289-317.
92. Singh, A. K. et al., “Photoactive bacteriorhodopsin variants”, 1997, Radiation Physics and Chemistry, vol. 49, No. 1, pp. 131-134.

I claim:

1. A method for producing biomass of photosynthesizing microorganisms/phototrophic algae and biomass of these microorganisms pigments, including the following stages:
irradiation of cultivated solution by natural sunlight (sun irradiation) and by additional electromagnetic radiation of low intensity within the frequency range 20-200 GHz in the bands of electromagnetic waves absorption by the chemical elements of the Periodic Table of elements or their compounds which are present in the environment (atmosphere), in particular atomic oxygen, atomic hydrogen and/or water vapor,
passing cultivated solution from the water treatment block to the microorganism selection separator with discharge return to the water intake post,
sending mature photosynthesizing microorganisms from this separator to bioreactor of cultivation,
irradiation of the cultivated solution in bioreactor of cultivation by applying natural sun lighting and electromagnetic irradiation of low intensity in the same frequency range,
separation of photosynthesizing microorganism biomass according to aging criterion of photosynthesizing microorganisms when they leave bioreactor of cultivation,
sending mature photosynthesizing microorganisms to the stress bioreactor where, e.g., photosynthesizing microorganisms are submerged into bidistilled water (or solution of another substance) which is preliminary irradiated by electromagnetic waves of low intensity in the same frequency range together with natural sun lighting,
irradiation of mature photosynthesizing microorganisms by natural sun lighting and electromagnetic radiation of low intensity in the same frequency range in microorganism stress bioreactor (tank),
sending the biomass of pigment from microorganism stress bioreactor output for centrifugation/flotation in the block of separating centrifuges,
drying of pigment biomass coming from output of block of separating centrifuges,
during drying the pigment biomass coming from output of block of separating centrifuges is irradiated by natural sun lighting and electromagnetic radiation of low intensity in the same frequency range,
sending cultivated photosynthesizing microorganism pigment biomass to the block of output control,
irradiation of cultivated photosynthesizing microorganism pigment biomass by electromagnetic radiation of low intensity in the same frequency range together with natural sun lighting,
packaging of cultivated photosynthesizing microorganism pigment biomass in the block of output control for delivery to consumers.

2. The method, according to claim 1, wherein the following is carried out:
directing of biomaterial discharge from sorting separator to bioreactor of cultivation,
irradiation of mature photosynthesizing microorganisms in microorganism stress bioreactor by additional electromagnetic radiation of low intensity in the frequency range 100-1000 GHz within the frequency bands of molecular spectra for electromagnetic waves radiation and absorption by the chemical elements which contain the pigment of cultivated photosynthesizing microorganism,
irradiation of pigment biomass coming from the output of the block of separating centrifuges during its drying process by additional electromagnetic radiation of low intensity in the frequency range 100-1000 GHz within the frequency bands of molecular spectra for electromagnetic waves radiation and absorption by the chemical elements which contain the pigment of cultivated photosynthesizing microorganism,
irradiation of cultivated photosynthesizing microorganism pigment biomass in the block of output control by additional electromagnetic radiation of low intensity in the frequency range 100-1000 GHz within the frequency bands of molecular spectra for electromagnetic waves radiation and absorption by the chemical elements which contain the pigment of cultivated photosynthesizing microorganism.

3. The method, according to claim 1, wherein the control for specific power of electromagnetic waves radiation is carried out within the limits of 0.05-10 mcWatt/cm².

4. The method, according to claim 1, wherein, during the process of cultivating photosynthesizing microorganisms/phototrophic algae and biomass of these microorganisms/phototrophic algae, modulation of electromagnetic radiation parameters is performed which is chosen out of group including amplitude modulation, frequency modulation, noise modulation, modulation of radiation pulses width and frequency and combination of the said modulation modes.

5. A device for producing biomass of photosynthesizing microorganisms/phototrophic algae and biomass of these microorganisms pigments, including:
post of water intake, water treatment block, bioreactor for bacteria cultivation, microorganisms stress bioreactor, block of raw material supply, bidistilled water source, block of separating centrifuges, drying block, packaging block, control block,
characterized by the fact that it is additionally equipped with
separator for selection,
separator for sorting,
generators of electromagnetic radiation of low intensity,
block of output control for biomaterials, whereas the output of the post of water intake in connected to the input of water treatment block,
which is connected to radiation generator, and its main output is connected to the input of separator for selection, the output of which is connected to input of bioreactor for cultivation,
the latter being connected to the output of the block of raw material supply and the output of radiation generator, and the output of the bioreactor of bacteria cultivation being connected to input of the separator for sorting,
the main output of which is connected to microorganisms stress bioreactor,
the latter being connected to output of the source of bidistilled water and the output of radiation generator, and the output of microorganisms stress bioreactor being connected to the block of separating centrifuges which is connected to the drying block by its main output,
the input of the latter being connected to output of radiation generator, and in the output it is connected to the input of the output control block,
which is connected to the output of radiation generator, and in its output is connected to the input of the packaging block,
discharge output from water treatment block is connected to output of discharge of the post for water intake,
discharge output from the separator for selection is connected to discharge output from the post of water intake,
discharge output from the separator for sorting is connected to input of bioreactor for cultivation,
output of low intensity electromagnetic radiation generator is connected to the source of bidistilled water,
discharge output of the block of separating centrifuges is connected to the input of the output control block.

6. The device, according to claim 5, wherein the control block is additionally equipped with registers for analog-to-digit transformations, registers for digit-to-analog transformations, decision block with digital elements of models for processes of cultivation and microorganisms stress, whereas outputs from control and measuring devices of the post of water intake, water treatment block, block of raw materials supply, bioreactors for cultivation and microorganisms stress, radiation generators, separator for selection, separator for sorting, block of separating centrifuges, drying block, block of output control and packaging block are connected to inputs of the registers for analog-to-digit transformations in control block,
outputs of register for analog-to-digit transformations are connected to inputs of digital elements of models for processes of cultivation and microorganisms stress,
outputs of digital elements of models for processes of cultivation and microorganisms stress are connected to inputs of digital elements in the decision block,
outputs of the latter being connected to inputs of register for digit-to-analog transformations in the control block,
control outputs of register for digit-to-analog transformations are connected to inputs of control schemes in the post of water intake, water treatment block, block of raw materials supply, bioreactors for cultivation and microorganisms stress, radiation generators, separators for selection and sorting, blocks of separating centrifuges, drying block, block of output control, packaging block.
